(19) United States
(12) Patent Application Publication
(10) Pub. No.: US 2026/0233246 A1
(43) Pub. Date: Aug. 13, 2026

(54) PIEZOELECTRICALLY OPERATED DISPENSER FOR DISPENSING LIQUID AND/OR VOLATILE SUBSTANCES AND METHOD

(71) Applicant: CTR, LDA, Samora Correia (PT)

(72) Inventors: Harold AUGIER, Zhongshan City (CN); Ren JIANHUI, Zhongshan City (CN); Luo MIN, Zhongsan (CN); Wei HONG, Zhongshan City (CN)

(21) Appl. No.: 19/157,445

(22) PCT Filed: Feb. 17, 2023
(86) PCT No.: PCT/EP2023/052332

§ 371 Date: Aug. 18, 2025

(51) Int. Cl. B05B 17/06

(52) U.S. Cl. CPC B05B 17/0607

(57) ABSTRACT

A piezoelectrically operated dispenser dispenses a liquid substance and has a housing and a container in which the substance is received. The container has a neck by which the container is detachably connectable to the housing. The dispenser contains a piezoelectrically operated atomizer system having a piezo element and a wick. The housing has a bottom wall which has a mounting dome which extends upwards and forms a mounting opening. The mounting opening merges into a wick opening at the end of the mounting dome, which is adjoined by the piezo element and also by a housing outlet opening. The mounting dome receives the neck of the container, which is introduced into the mounting dome via the mounting opening, and holds it releasably in the mounting dome by a holding device such that the wick extends with its free wick end through the wick opening and adjoins the piezo element.

The invention relates to a piezoelectrically operated dispenser for dispensing liquid and/or volatile substances according to the preamble of claim 1. Furthermore, the invention relates to a method for operating a piezoelectrically operated dispenser for dispensing liquid and/or volatile substances according to the preamble of claim 29.

Dispensers are generally used to distribute a liquid active substance, such as a perfume, insecticide or other substance. It is also generally known to convert the substances into fine or extremely fine particles or droplets by means of a piezoelectrically operated dispenser, which has a piezoelectrically operated atomizer system, and to deliver them to the environment as a spray mist. A piezoelectrically operated atomizer system is understood to be an atomizer system with a piezo element that utilizes the piezoelectric effect, for example, by applying an electrical voltage to execute a mechanical movement or oscillation (piezoelectric actuator), by means of which mechanical movement or oscillation the droplets are then generated. This is generally known and does not need to be explained further.

The liquid or volatile substances to be dispensed by means of the dispenser are regularly held in a container which is inserted into a housing of the dispenser in order to guide the substance via a wick to the piezoelectrically operated atomizer system. These delivery systems are often very error-prone and therefore often lead to problems with the spraying performance of the dispensers.

It is therefore an object of the invention to create a piezoelectrically operated dispenser for dispensing liquid and/or volatile substances, with which the substance can be dispensed in a functionally safe and reliable manner, even over a long period of time.

Furthermore, it is an object of the invention to provide a method by means of which substance dispensing in a room is optimized for the user, can be adjusted flexibly and individually and, above all, also helps to avoid a rapid habituation effect.

This object is solved with the features of the independent patent claims. Advantageous embodiments are the subject of the related subclaims.

According to claim 1 a piezoelectrically operated dispenser for dispensing liquid and/or volatile substances is provided. This dispenser has a housing designed as a hollow body, which has a cavity in its interior as a housing interior.

Furthermore the dispenser has a container in which a substance to be dispensed and/or volatilized is received, the container having a neck as an outlet opening designed in the manner of a pipe socket and/or in the manner of a bottle neck, by means of which the container is detachably connectable to the housing, the housing and the container being arranged above one another in the assembled state in such a way that the housing, viewed in the vertical axis direction, is arranged above the container.

The dispenser further comprises a piezoelectrically operated atomizer system, by means of which the substance taken from the container is atomized, the atomizer system having an electrically actuable piezo element and a wick, by means of which the substance received in the container is feedable to the piezo element.

Furthermore, in the assembled state of the container, the wick projects with a first partial region into the substance received in the container and projects with an opposite free end out of the neck of the container.

According to the invention, the housing has a housing bottom wall on its underside in relation to the vertical axis direction. This housing bottom wall has, preferably in a central region, a mounting dome which extends upwards in the vertical axis direction and inwards into the housing interior and forms a mounting opening on the bottom wall side which is accessible from the housing exterior.

Furthermore, the mounting opening in the interior of the housing merges into a wick opening at the end of the mounting dome, which is adjoined, with respect to the vertical axis direction, outwardly and/or upwardly, by the piezo element and also by a housing outlet opening, via which a substance to be discharged escapes from the housing into the environment.

The mounting dome is suitable and designed to receive the neck of the container, which is introduced into the mounting dome via the mounting opening, at least partially, preferably completely, and to hold it releasably in the mounting dome by means of a holding device in such a way that the wick extends with its free wick end in the assembled state of the container, preferably with formation of a circumferential gap distance from a wick opening wall region forming the wick opening, into and/or through the wick opening and adjoins the piezo element, preferably adjoins the piezo element and is in contact with the piezo element.

This leads not only to a compact, relatively small construction of the dispenser, which is safe and easy to handle and operate, but also to a construction which allows the wick to be brought into such a stable and optimized position in relation to the piezo element that a permanently good and functionally reliable substance dispensing can take place via the wick to the piezo element.

This is achieved in a particular way with a preferred embodiment in which it is provided that the mounting dome has a mounting and receiving zone for the piezo element adjoining the wick opening, which mounting and receiving zone is suitable and designed to arrange and/or hold the piezo element in the area of the wick opening, so that the wick adjoins the piezo element with its free wick end, preferably adjoins the piezo element and is in contact with the piezo element. In a further preferred embodiment said mounting and receiving zone has a receiving chamber for the piezo element which, for substance discharge, is provided with at least one receiving chamber outlet opening, preferably formed at the free end of the mounting dome, and in which the piezo element is received such that it is displaceable into different positions, preferably, when seen in the direction of the vertical axis, is received in such a way that it is displaceable into different positions. Said mounting and receiving zone also has a, preferably elastic, pretensioning device by means of which the piezo element is pretensionable, preferably elastically pretensionable, for contact with the free wick end, preferably in such a way that, during the mounting of the container, the free wick end is bringable into contact with the piezo element and, as a result, the piezo element is displaceable against the force of the pretensioning device in the receiving space, in particular is displaceable as seen in the vertical axis direction.

Especially with such a design, a particularly functionally safe and reliable positioning and contact of the piezo element on the wick or the wick on the piezo element is achieved, since the displacement option of the piezo element means that the piezo element can always be displaced to the desired position with respect to the wick with the pretensioning device, even with different wick lengths. Tolerance-related construction differences can therefore no longer have a negative effect on the spray performance.

According to a further preferred embodiment, the piezo element is disk-like and is, aligned transversely with respect to the vertical axis direction, accommodated in the receiving chamber, preferably with a piezo element disk edge region projecting beyond the free wick end in the assembled state, as seen in the circumferential direction. With such a construction, a particularly advantageous areal contact of the free wick end with the piezo element can be achieved, which leads to a functionally reliable supply of the piezo element with the substance to be dispensed. This is especially the case, when, according to another preferred embodiment, the free wick end contacts the piezo element and/or a central area of the piezo element with its, preferably flat, front side.

According to another preferred embodiment which allows a simple manufacturing, the receiving chamber is formed by at least one receiving chamber wall which extends away from the wick opening in the vertical axis direction and at least partially, preferably completely, encloses the wick opening, as seen in the circumferential direction of the wick opening. In this regards it is further preferred that the receiving chamber wall encloses the wick opening at a distance from the wick opening edge and/or is tubular.

In addition, a compact and simple structure results with a construction with which the receiving chamber wall, at an end of the receiving chamber opposite the wick opening and thus at the free end of the mounting dome, forms the receiving chamber outlet opening which, with respect to the vertical axis direction, is in alignment with the wick opening and/or the housing outlet opening.

In principle, there are many different ways of designing the pretensioning device to ensure reliable pretensioning and thus contact between the piezo element and the wick. According to a preferred embodiment that is simple in terms of production technology and functionally reliable, this is achieved by a pretensioning device that is formed by an elastic spring element, preferably by an elastic compression spring, which on the one hand acts indirectly or directly on the piezo element and on the other hand is supported on the mounting dome, preferably supported in the region of the receiving chamber, preferably the elastic spring element being formed by a helical spring or by a disc spring assembly.

For a compact design it is preferred that the pretensioning device is also arranged in the receiving chamber and/or that the pretensioning device is supported in the region of the receiving chamber outlet opening by means of a supporting device. Said supporting device is preferably formed by a fixing sleeve, in particular a cap, which is placed on the free end of the mounting dome, preferably releasably and/or held by means of a clamping and/or frictional and/or form fit, and which covers the receiving chamber outlet opening with a sleeve wall. To ensure that the substance can escape from the receiving chamber a sleeve opening is formed in the sleeve wall, via which the substance to be dispensed escapes from the receiving chamber. In addition, the opening edge region of the sleeve opening projects into the receiving chamber outlet opening for supporting the pretensioning device, preferably with the fixing sleeve being U-shaped in cross section and the sleeve wall being formed by the U-base, while the U-legs engage around the mounting dome on its outer side.

The advantage of this solution with an attachable fixing sleeve is clearly that it significantly simplifies the assembly and also the disassembly of the pretensioning device in conjunction with the piezo element. The fixing sleeve is a separate component that can be easily fitted and removed. In principle, however, the supporting device can of course also be designed in other ways, e.g. integrally attached to the opening edge region of the receiving chamber outlet opening, e.g. as an integral projection projecting into the opening region.

For a particularly secure support of one end of the pretensioning device, it is further advantageous when the opening edge region of the sleeve opening projecting into the receiving chamber outlet opening, as seen in the circumferential direction, has at least in some regions a bend directed inwards into the receiving chamber in the direction of the vertical axis, so that between the bend and the associated wall region of the receiving chamber inner wall there is formed a supporting device which is U-shaped in cross section and in which the pretensioning device, preferably a spring element with its one spring end, is received and supported.

To ensure an unhindered escape of the substance it is furthermore preferred that the opening cross-section of the sleeve outlet opening is larger than the cross-section of the free wick end adjacent to the piezo element and/or corresponds approximately to the opening cross-section of the housing outlet opening.

A particularly advantageous unhindered outlet is also achieved in that the housing outlet opening, viewed in the vertical axis direction, is located above and/or in alignment with the free wick end and/or in alignment with the wick opening, preferably in alignment with the wick opening and the receiving chamber outlet opening and optionally the sleeve outlet opening.

A particularly advantageous unhindered outlet is also achieved by means of another optional embodiment according to which the free end of the mounting dome in the housing interior is directly adjacent to the housing outlet opening and/or in that the mounting dome, viewed in the vertical axis direction, extends from the bottom wall essentially over the entire height of the housing interior to the housing outlet opening.

For the construction of the mounting dome, there are again numerous different possibilities. A particularly advantageous compact, small-scale and functionally integrated structure, which is also easy to manufacture, is obtained with a particularly preferred embodiment, according to which the mounting dome has, as seen in the vertical axis direction, an upper dome portion and, in relation thereto, a lower dome portion. Said lower dome portion forms the mounting opening and the holding device for the neck of the container and/or extends approximately to the center of the housing interior, as viewed in the vertical axis direction. Said upper dome portion has the mounting and receiving zone for the piezo element and the wick opening, the wick opening being in the form of a wick channel which has a lower opening area, relative to the vertical axis direction, via which the wick is insertable into the wick channel, and, in contrast thereto, an upper opening area, it being preferably provided that the free wick end projects beyond the upper opening area in the mounted state and projects into the receiving chamber of the mounting and receiving zone.

In an optimized embodiment in which the above objectives are achieved even better, it is provided that the wick channel, viewed in the vertical axis direction, directly adjoins the mounting opening of the lower dome portion in an upward direction and/or that an end face of the neck of the container in the assembled state abuts and/or is supported against a wall portion of the upper dome portion forming the lower opening area of the wick channel.

A simple and functionally reliable construction of a holding device that is easy and quick to operate is achieved with a construction which provides that the holding device for releasably holding the container in the mounting dome is formed by a snap connection, preferably in such way that the snap connection is formed by at least one snap hook which is elastically resiliently arranged on the mounting dome and has at least one latching projection, preferably arranged on the end side, which, in the case of a mounted container, engages behind a neck-side retaining region or engages in a neck-side retaining region, preferably in such way that the neck of the container has on its outer side an at least sectionally, preferably annular, circumferential bead or an at least sectionally, preferably annular, circumferential groove as retaining region.

As the inventor's experiments have shown, it is particularly advantageous for a functionally reliable connection when a plurality of snap hooks are arranged and formed in the lower dome portion forming the mounting opening, which snap hooks, in the inserted state of the container, are arranged annularly and spaced apart from one another around the neck of the container and engage with their respective latching projections behind the retaining region or engage in the retaining region.

In order to ensure that no unwanted access to the interior of the housing can take place, it is particularly advantageous that the housing is substantially closed at its underside, referred to the vertical axis direction, and the housing bottom wall is formed as a continuous housing bottom wall closing or shutting the housing at the bottom. A further advantage of such a housing bottom wall can be seen in the fact that the housing can then be very well supported on the container. The housing bottom wall can either be an integral housing component or, in principle, also be formed by a separate housing component which is to be connected to the housing.

According to another preferred embodiment, which is easy to manufacture and offers an optimized compact overall design with a very valuable appearance, it is provided that the housing bottom wall forming the underside of the housing surrounds the, preferably centrally arranged, mounting dome with a bottom wall edge region which is aligned essentially horizontally with respect to the vertical axis direction and seen in the circumferential direction.

A compact and high-quality housing design is achieved with a design where it is provided that a housing side wall adjoins the outer edge region, viewed in the radial direction, of the housing bottom wall, which housing side wall extends upwards to form a hollow body and merges into a housing top wall located at a distance above the housing bottom wall, in which housing top wall the housing outlet opening is formed, preferably in such way that the housing side wall merges smoothly and/or with a curve into the housing top wall. According to a particularly preferred embodiment in this regard provides that the housing has a bulbous and/or hat-like and/or spherical segment-like shape which is closed at the bottom by the substantially horizontally oriented housing bottom wall.

Furthermore, a compact and high-quality housing design is achieved with a design where it is provided that the housing, with respect to a plan view, has substantially the same outer circumference and/or the same circumferential geometry as the container, preferably in such way that the housing and the container have a substantially circular outer geometry.

And consequently, a compact and high-quality container design is achieved with a design where it is provided that the container has a cylindrical circumferential wall with a container bottom wall extending substantially horizontally with respect to the vertical axis direction and a container top wall extending substantially parallel thereto, the container top wall having, preferably centrally, the neck. With such a construction of the container, a particularly preferred design is achieved in which, in the assembled state, the housing together with the container forms a tower-like structure, the housing resting with its housing bottom wall on the container top wall, in particular the housing resting and being supported with its housing bottom wall at least in regions on the container top wall.

To ensure a safe and reliable operation of the dispenser, a control device is arranged in the housing, which is connected, preferably electrically, to the atomizer system for controlling the piezo element, and/or to an indicator display, which is arranged on the housing side and is visible from outside the housing, for displaying the set dispensing rate, and/or to an activation and deactivation device, which is arranged on the housing side and is accessible from outside the housing, for activating and deactivating the dispenser, and/or to a setting device, arranged on the housing side and accessible from outside the housing, for setting the dispensing rate, and/or to an electrical connection element, arranged on the housing side and accessible from outside the housing, in particular a USB socket, and/or to a charging indicator, arranged on the housing side and visible from outside the housing, for indicating the charging state of an energy storage device, in particular a battery or an accumulator, of the dispenser, preferably in such way that the activation and deactivation device for activating and deactivating the dispenser simultaneously also forms the setting device for setting the dispensing rate.

Furthermore, it is preferred that a printed circuit board assembly of the control device is arranged in the housing, preferably in the area of the housing bottom wall, which is electrically coupled to the piezo element and/or to the indicator display and/or to the activation and deactivation device and/or to the setting device and/or to the electrical connection element and/or to the charging indicator. That means that the control device comprises a circuit board on which different electronic components are arranged. The components on the board are electrically connected to each other by means of a suitable board layout on the board in order to provide the desired electronic functionality of the piezo element and thus of the dispenser. For this purpose, the circuit board has a plurality of electrical conductors which correspond to the circuit board layout described above.

To ensure a secure and reliable holding of the wick it is provided that a tubular wick holding device is inserted in the neck of the container, by means of which the wick is held tightly and/or precisely in position in the container.

The object as regards the method is solved with the features of independent method claim providing a method for operating a dispenser according to which the activation and deactivation device for activating and deactivating the dispenser simultaneously also forms the setting device for setting the dispensing rate. According to this method the piezoelectrically operated atomizer system is actuated such that, starting from a switched-off state of the dispenser, a first actuation of the activation and deactivation device for a defined minimum switch-on actuation period (preferably lasting about 0.1 to 3 seconds) switches on the dispenser and sets it to a first operating mode with which a first dispensing rate is set. Subsequently, there a two alternative options for operating operate the dispenser:

The first option is characterized in that subsequently, for each further actuation of the activation and deactivation device which is shorter than a defined minimum switch-off actuation period (preferably lasting about 0.1 to 3 seconds), at least one further operating mode with a higher dispensing rate than the respective previous dispensing rate is set, and in that the activation and deactivation device is switched off in the event of an actuation which exceeds a defined switch-off actuation period.

The second option, which is an alternative to the aforementioned first option, is characterized in that subsequently, for each further actuation of the activation and deactivation device, a defined number of further operating modes, preferably two further operating modes, with a higher dispensing rate than the respective previous dispensing rate is set, and in that after the actuation of the last operating mode a further actuation of the activation and deactivation device switches off the activation and deactivation device.

This results in an advantageous integration of functions, in which the activation and deactivation device simultaneously forms the setting device in an advantageous, component-saving double function. Such a functional integration is also technically simple to manufacture and easy for the operator to learn.

Even though these two options are presented as alternatives here, it is in principle possible to shift or reprogram the dispenser such that the same dispenser can be used to switch back and forth between the two options, i.e. both options can be offered on the same unit.

The above advantages are all the more true if, in a preferred embodiment, it is provided that the minimum switch-on actuation period and the minimum switch-off actuation period are substantially equal in length, preferably each lasting about 0.1 to 3 seconds.

According to a further preferred embodiment of the first option, it is, for example, particularly advantageous for simple and intuitive operation if it is provided that, after reaching the highest intended dispensing rate and a further actuation of the activation and deactivation device, which is shorter than the minimum switch-off actuation period, the first, lowest dispensing rate is set again.

A very simple operation with a technically easy controllable and producible increase of the dispensing rate results with a method in which, after switching on the dispenser and setting the first dispensing rate, a maximum of two further dispensing rates corresponding to a maximum of three operation modes are settable.

According to another preferred embodiment the spraying time of the dispenser, in the first operating mode is in each case 1 to 10 seconds, preferably in each case 2 to 5 seconds, most preferably in each case about 3 to 4 seconds, the interval between the individual spraying times being 5 to 30 minutes, preferably 10 to 20 minutes, most preferably about 15 to 17 minutes. Furthermore, in at least one further operating mode, the spraying time of the dispenser is increased compared to the spraying time of the first operating mode and/or the interval between the individual spraying times is shortened compared to the interval of the first operating mode. With such times, optimized substance dispensing with a desired dose that can be perceived by the user according to his presetting is achieved in an effective manner.

As tests by the inventors have shown, optimized operation is further achieved when, in a second adjustable operating mode, the spraying time of the dispenser is increased compared to the spraying time of the first operating mode by 1 to 3 seconds, preferably by in about 1 second, and/or the interval between the individual spraying times is 10 to 25 minutes, preferably 15 to 20 minutes, most preferably in about 17 minutes.

And furthermore in that in a third adjustable operating mode the spraying time of the dispenser is increased compared to the spraying time of the second operating mode by 1 to 3 seconds, preferably by in about 1 second, and/or the interval between the individual spraying times is 2 to 10 minutes, preferably 3 to 8 minutes, most preferably in about 5 minutes.

An advantageous, automatic operation is achieved with a method of operation according to which it is provided that the piezoelectric atomizer system, after its activation, is operated in an activation mode for a defined activation time, preferably for 6 to 12 hours, most preferably for about 8 to 9 hours, before it automatically switches to a sleeping mode for the remaining time, based on a period greater than the activation time, preferably based on 24 hours, and that the piezoelectric atomizer system automatically switches back to the activation mode after the time for the sleeping mode has elapsed.

The invention is explained in more detail below with reference to solely exemplary figures.

The figures show:

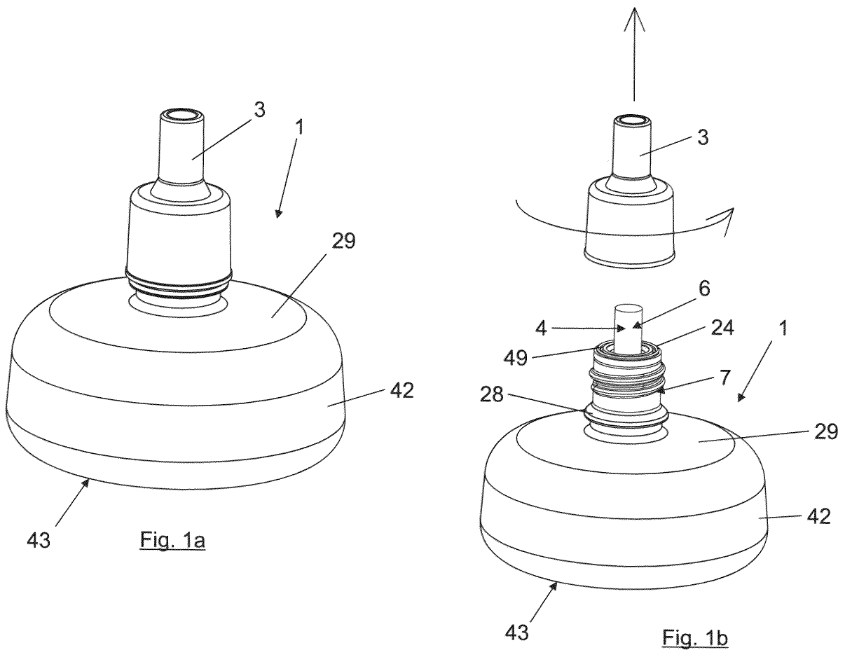

FIG. 1a shows a schematic perspective view of a container designed refill in the closed state, as a

FIG. 1b the container according to FIG. 1a with the closure cap unscrewed,

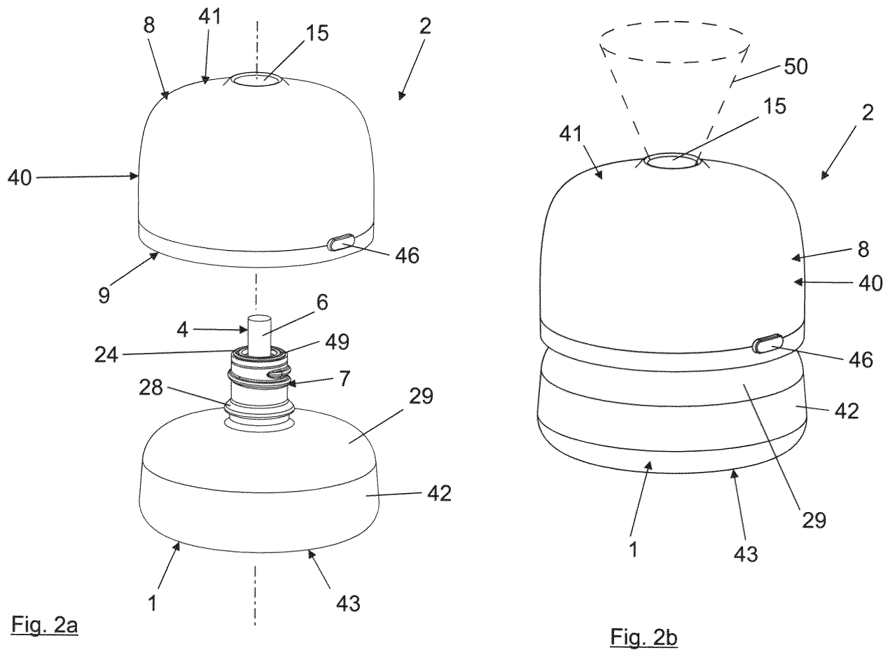

FIG. 2a the container as shown in FIG. 1b with the closure cap unscrewed shortly before a housing of a piezoelectrically operated dispenser is fitted,

FIG. 2b the piezoelectrically operated dispenser in the assembled state, with the housing detachably connected to the container,

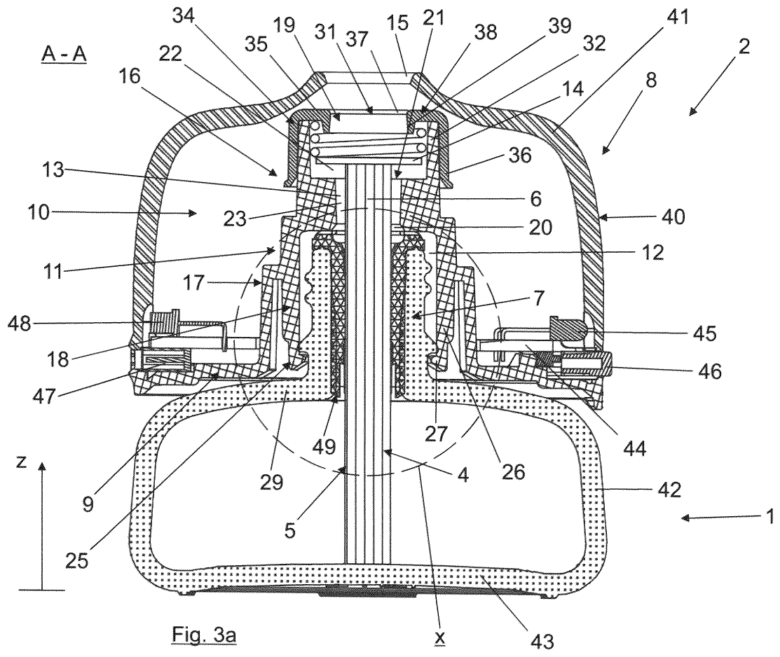

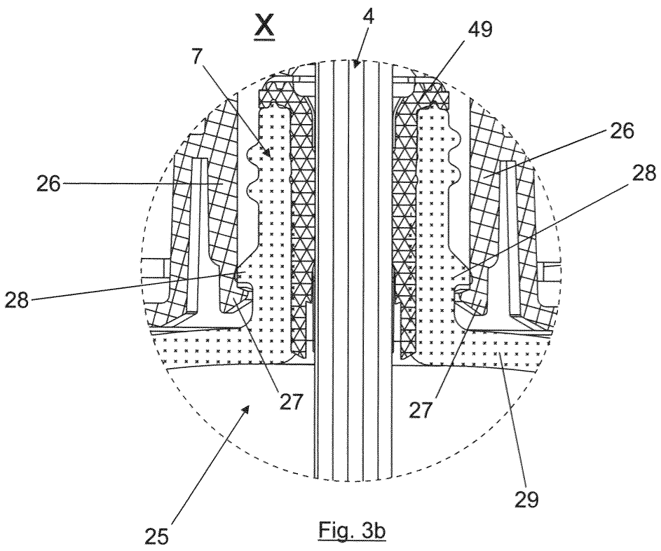

FIG. 3a a schematic cross-section along line A-A of FIG. 2b,

FIG. 3b an enlarged view of detail X of FIG. 3a.

FIG. 1a shows a container 1, also referred to as a refill, which is filled with a preferably liquid substance to be dispensed into the environment, for example a living space, by means of a piezoelectric operated dispenser 2 shown in FIGS. 2a and 2b.

In FIG. 1a, the container 1 is still closed by means of a closure cap 3, which in FIG. 1b, however, has already been unscrewed, so that a wick 4 is visible, which projects with a first section 5 (see also FIG. 3a) into the substance contained in the container 1 and with an opposite free wick end or free end 6 from a neck 7 of the container 1.

The neck 7 is formed here in the manner of a pipe socket and/or in the manner of a bottle neck and serves to detachably connect the container 1 to a housing 8 of the dispenser 2 in a manner to be described.

This connection between the housing 8 and the container 1 is clearly shown in FIGS. 2a and 2b, with FIG. 2b showing the fully assembled state of the dispenser 2.

In FIG. 3a, which shows a section along the line A-A of FIG. 2b, the connection between the container 1 and the housing 8 is now described in more detail, also in connection with FIG. 3b, which shows an enlarged detail X of FIG. 3a, as is the further basic and exemplary construction of the dispenser 2 as a whole:

As can be seen in particular from FIG. 3a, the housing 8 has a housing bottom wall 9 on its underside, referred to the vertical axis direction z. Accordingly, the housing 8 is essentially closed on its underside, relative to the vertical axis direction z, and the housing bottom wall 9 is preferably and exemplarily designed here as a continuous housing bottom wall 9 closing the housing 8 at the bottom.

This housing bottom wall 9 has, here preferably and by way of example in a central region, a mounting dome 11 extending upwards in the vertical axis direction z and inwards into the housing interior 10, which forms a mounting opening 12 on the bottom wall side, which is accessible from the outside of the housing.

In the housing interior 10, this mounting opening 12 merges into a wick opening 13 at the end, which is connected to a piezo element 14 on the outside or at the top, in relation to the vertical axis direction z, and also to a housing outlet opening 15, through which a substance to be dispensed can escape from the housing 8 into the environment.

The mounting dome 11 has an upper dome portion 16, relative to the vertical axis direction z, and a lower dome portion 17. As can be seen in particular from FIG. 3a, the lower dome portion 17 forms the mounting opening 12 as well as a holding device 18 for the neck 7 of the container 1, whereby the lower dome portion 17 in the example case shown here also extends approximately to the middle of the housing interior 10, as seen in the vertical axis direction z.

The upper dome portion 16 here has a mounting and receiving zone 19 for the piezo element 14 and the wick opening 13, the wick opening 13 being designed here as an example as a wick channel which has a lower opening area 20, relative to the vertical axis direction z, via which the wick 4 or its free end 6 can be inserted into the wick channel in a manner to be described.

In addition, the wick channel has an upper opening area 21 located above the lower opening area 20. As is preferred in the example shown here, the free wick end 6 projects beyond the upper opening area 21 in the assembled state of the container 1 and projects into a receiving chamber 22 of the mounting and receiving zone 19.

As can be seen from FIG. 3a, the free end 6 of the wick 4 extends into and/or through the wick opening 13, which is formed here as a wick channel, in such a way that the free wick end 6 has a circumferential gap distance 23 from a wick opening wall region forming the wick channel.

As can be seen from FIG. 3a, the wick channel, viewed in the vertical axis direction z, directly adjoins the mounting opening 12 of the lower dome portion 17 at the top, the structure and arrangement being selected in such a way that a free end face 24 of the neck 7 of the container 1 in the assembled state rests and is supported against a wall region of the upper dome portion 16 forming the lower opening area 20.

The holding device 18 for releasably holding the container 1 or the neck 7 in the mounting dome 11 is formed here by a snap connection 25. This snap connection 25 has, as an example, a plurality of snap hooks 26 in the lower dome portion 17 forming the mounting opening 12 (see also FIG. 3b), which in the inserted state of the container 1 are arranged annularly and spaced apart from one another around the neck 7 of the container 1 and engage with their respective latching projections 27 behind a retaining region 28, which in this case is formed as an annular circumferential bead on the neck 7.

When the housing 8 is fitted or the container 1 is inserted, the neck 7 of the container 1 is inserted into the mounting opening 12 of the mounting dome 11, with the retaining region 28, which is designed as a bead, forcing the elastically resiliently connected snap hooks 26 outward. For this purpose, both the bead of the retaining region 28 and the latching projections 27 preferably have corresponding sliding and run-up chamfers.

As soon as the neck 7 is inserted into the mounting opening 12 to such an extent that the retaining region 28, viewed in the vertical axis direction z, comes to rest above the latching projections 27, the elastically resiliently connected snap hooks 26 can spring back again so that they engage with their latching projections 27 behind the retaining region 28, which is designed as a bead. During disassembly, this process is reversed accordingly.

As can also be seen from FIG. 3a, a tubular wick holding device 49 is inserted in the neck 7 of the container 1, by means of which the wick 4 is held tightly and precisely in position in the container 1.

As can be seen from FIG. 3a, both the length of the neck 7 and the length of the mounting opening 12, in each case viewed in the vertical axis direction z, are designed and dimensioned in such a way that not only the end face 24 of the neck rests against the wall region of the upper dome portion 16 forming the lower opening area 20, but the housing bottom wall 9 is directly adjacent to a container top wall 29 or, if necessary, is even supported at least in partial regions on the container top wall 29. This is then done essentially with a substantially horizontally aligned bottom wall edge region 30, which surrounds the mounting dome 11 arranged centrally here in the circumferential direction.

However, before the outer geometry of the container 1 or housing 8 is discussed in more detail, the structure of the mounting and receiving zone 19 will be explained in more detail below:

As already shown, the mounting opening 12 in the housing interior 10 merges into the wick opening 13, which is designed here as a wick channel and to which the piezo element 14, which is arranged and received in the receiving chamber 22 of the mounting and receiving zone 19, is connected outwardly and upwardly with respect to the vertical axis direction z.

For substance delivery, the receiving chamber 22 is provided with a receiving chamber outlet opening 31 formed at the free end of the mounting dome 11 and formed here by a tubular receiving chamber wall 32, which encloses the wick opening 13 or its upper opening area 21 at a distance from the wick opening edge. Accordingly, the receiving chamber outlet opening 31 is also formed at an end of the receiving chamber 22 remote from the wick opening 13 or the upper opening area 21 and thus at the free end of the mounting dome 11, the receiving chamber outlet opening 31 being aligned with the wick opening 13 and the housing outlet opening 15.

In this receiving chamber 22, which extends over a predetermined distance in relation to the vertical axis direction z, the piezo element 14, which is exemplary here in the form of a disk, is now accommodated and aligned transversely to the vertical axis direction z. The piezo element 14 can also be moved into different positions in the vertical axis direction z. In addition, the piezo element 14 can be displaced into different positions as viewed in the vertical axis direction z.

In order to carry out this displacement, an elastic spring element 33 is also provided, which is preferably formed by an elastic compression spring, which on the one hand engages on the edge of the piezo element 14 and on the other hand is supported in the area of the receiving chamber outlet opening 31 in a manner to be described. Specifically, the elastic spring element 33 is formed here by a helical spring, for example, but could also be formed by a disk spring assembly.

This elastic spring element 33 acts as a pretensioning device and causes the piezo element 14 to come into flat contact with the flat end face of the free wick end 6 in the manner shown in FIG. 3a, with the free wick end 6 also resting in a central region of the piezo element 14.

Such a structure ensures that the free wick end 6 can be brought into contact with the piezo element 14 during assembly of the container 1 and that the piezo element 14 can thereby be displaced against the force of the elastic spring element 33 in the receiving chamber 22. This ensures that, irrespective of any tolerances, a functionally safe and reliable contact of the free wick end 6 with the piezo element 14 is guaranteed and, accordingly, an optimized operation of the piezoelectrically operated atomizer system is possible, which is essentially formed by the electrically actuable piezo element 14 and the wick 4.

Specifically and merely by way of example, the support in the area of the receiving chamber outlet opening 31 is implemented here by a fixing sleeve 34, which is designed here as a cap and is placed on the free end of the mounting dome 11 in such a way that it covers the receiving chamber outlet opening 31 with a sleeve wall 35.

The fixing sleeve 34 is preferably detachable here, but is held on the free end by means of a clamping and/or frictional and/or positive connection, in such a way that the fixing sleeve 34, which is U-shaped in cross section, engages with its U-legs 36 around the outside of the mounting dome 11.

To allow the substance to be dispensed to escape from the receiving chamber 22, a sleeve opening 37 is formed in the sleeve wall 35, the opening edge region of which projects into the receiving chamber outlet opening 31 to support the elastic spring element 33. This opening edge region 38 has here, by way of example, as seen in the circumferential direction, a bend 39 directed inwardly in the vertical axis direction z into the receiving chamber 22, so that between the bend 39 and the associated wall region of the receiving chamber inner wall there is formed a supporting device of U-shaped cross-section, in which the elastic spring element 33 is received and supported with its one spring end.

As can be seen in FIG. 3a, the opening cross-section of the sleeve opening 37 is preferably larger than the cross-section of the free wick end 6 adjacent to the piezoelectric element and corresponds approximately to the opening cross-section of the housing outlet opening 15.

As can be seen from FIG. 3a, the housing outlet opening 15, viewed in the vertical axis direction z, is located above and in alignment with the free wick end 6, the wick opening 13, the receiving chamber outlet opening 31 and the sleeve opening 37.

In addition, it can be seen from FIG. 3a that the mounting dome 11 with its free end in the housing interior 10 is directly adjacent to the housing outlet opening 15 and that the mounting dome 11, viewed in the vertical axis direction z, extends from the housing bottom wall 9 essentially over the entire height of the housing interior 10 to the housing outlet opening 15.

As can be seen from FIG. 3a, the outer edge region of the housing bottom wall 9, viewed in the radial direction, is adjoined by a housing side wall 40 which extends upwards to form a hollow body and merges into a housing top wall 41 located at a distance above the housing bottom wall 9, in which the housing outlet opening 15 is formed.

As can be seen from FIGS. 2a, 2b and 3a, the housing side wall 40 in the present embodiment is rounded and merges smoothly with the housing top wall 41.

The housing 8 here also has a bulbous or spherical segment-like shape which, as already explained, is closed at the bottom by the essentially horizontally oriented housing bottom wall 9.

The housing 8 further has, with respect to a top view, essentially the same outer circumference or the same circumferential geometry as the container 1, in the example case shown here an essentially circular outer geometry.

The container 1 itself has a cylindrical circumferential wall 42 with a container bottom wall 43 extending substantially horizontally with respect to the vertical axis direction z and the container top wall 29 extending substantially parallel thereto, which has already been described above.

Accordingly, in the assembled state, the housing 8 forms a tower-like structure together with the container 1, with the housing 8 resting with its housing bottom wall 9 on the container top wall 29.

Furthermore, a printed circuit board assembly 44 with a control device is arranged in the housing 8 in the area of the housing bottom wall 9, which is electrically coupled to the atomizer system for controlling the piezo element 14. In addition, the control device of the printed circuit board assembly 44 is coupled to an indicator display 45 arranged on the housing side and visible from outside the housing for displaying the set delivery rate, which is formed here, for example, by one or more LEDs.

Furthermore, the control device is electrically coupled to an activation and deactivation device 46 (for example a push or press button), by means of which the dispenser 2 can be activated or deactivated, i.e. switched on or off. Here again, activation and deactivation device 46 is arranged on the housing side and is accessible from outside housing 8.

This activation and deactivation device 46 also forms a setting device for setting the delivery rate, which can be realized, for example, in such a way that the activation and deactivation device 46 must be actuated several times in succession in order to achieve the individual settings.

Furthermore, the control device is electrically coupled to an electrical connection element, preferably a USB socket 47, arranged on the housing side and accessible from outside the housing 8, and to a charging indicator 48, arranged on the housing side and visible from outside the housing 8, for indicating the charging state of a battery of the dispenser 2, for example.

FIG. 2b also shows, merely by way of example and schematically, that the dispenser 2 according to the invention can be used to obtain a spray mist 50 which can reach a plume height of 10 to 20 cm, for example.

For example, an exemplary embodiment of a dispenser, as described above, may be operated as follows:

In order to activate the dispenser, starting from a switched-off position, a first actuation of the activation and deactivation device for a defined minimum switch-on actuation period, preferably lasting about 0.1 to 3 seconds, is made which activates the dispenser and sets it to a first operating mode (named “Low”) with which a first dispensing rate is set.

If it is desired to set a higher dispensing rate a further actuation of the activation and deactivation device which is shorter than a defined minimum switch-off actuation period is made in order to set at least one further operating mode with a dispensing rate which is higher than the respective previous dispensing rate. Preferably the dispenser comprises two further higher dispensing rates (named “Medium” and “High”) corresponding to in total three operation modes (named “Low”, “Medium” and “High”).

After reaching the highest dispensing rate (named “High”), there are basically two options: Firstly, the first, lowest dispensing rate (“Low”) can be set again by a further actuation of the activation and deactivation device, which is shorter than the minimum switch-off actuation period, or, alternatively, the activation and deactivation device respectively the dispenser can be switched off.

From the above it is also clear that, according to the first one of the two options, the activation and deactivation device is switched off in the event of an actuation which exceeds a defined switch-off actuation duration.

That means that the dispenser could in principle offer the following two ways of operation (“press” refers here to the pressing of an exemplary press button as actuation and deactivation device 46):

First way of operation:

$${1}^{s t} press = On / Low$$

$${2}^{n d} press = Medium$$

$${3}^{r d} press = High$$

$${4}^{t h} press = Off$$

Second way of operation:

$${1}^{st} long press = On / Low$$

$${2}^{st} long press = Medium$$

$${3}^{st} long press = High$$

$${4}^{st} long press = Low$$

$${5}^{st} long press = Medium$$

$${6}^{st} long press = High$$

$${7}^{st} long press = Low$$

. . . and so on until we have another long press to switch off.

Further details for an exemplary operation of the dispenser can be found in the table below:

between

| | | Interval between | |
|---|---|---|---|
| | Spraying time | each spray | Working |
| Setting | (+/−5%) | (+/−5%) | Time |
| Low | 3 seconds | 17 minutes | 8 hours |
| Medium | 4 seconds | 12 minutes | |
| High | 5 seconds | 5 minutes | |
| Sleeping | OFF | OFF | After 8 hours |
| Mode | | | to 24 hours |
| Off | OFF | OFF | |

### LIST OF REFERENCE SIGNS

1 container
2 dispenser
3 closing cap
4 wick
5 first section
6 free end
7 neck
8 housing
9 housing bottom wall
10 housing interior
11 mounting dome
12 mounting opening
13 wick opening
14 piezoelectric element
15 housing outlet opening
16 upper dome portion
17 lower dome portion
18 holding device
19 mounting and receiving zone
20 lower opening area
21 upper opening area
22 receiving chamber
23 gap distance
24 front side
25 snap connection
26 snap hook
27 latching projection
28 retaining region
29 container top wall
30 bottom wall edge region
31 receiving chamber outlet opening
32 receiving chamber wall
33 elastic spring element
34 fixing sleeve
35 sleeve wall
36 U-legs
37 sleeve opening
38 opening edge region
39 bend
40 housing side wall
41 housing top wall
42 circumferential wall
43 container bottom wall
44 printed circuit board assembly
45 indicator display
46 activation and deactivation device
47 USB socket
48 charging indicator
49 wick holding device
50 spray mist

1-36. (canceled)

37. A piezoelectrically operated dispenser for dispensing liquid and/or volatile substances, comprising:
a housing configured as a hollow body and having a cavity in its interior defining a housing interior and a housing outlet opening formed therein;
a container in which a substance to be dispensed and/or volatilized is received, said container having a neck as an outlet opening configured in a manner of a pipe socket and/or in a manner of a bottle neck, by means of said neck said container is detachably connectable to said housing, said housing and said container being disposed above one another in an assembled state such that said housing, viewed in a vertical axis direction, is disposed above said container;
a piezoelectrically operated atomizer system, wherein by means of said piezoelectrically operated atomizer system the substance is taken from said container and is atomized, said piezoelectrically operated atomizer system having an electrically actuable piezo element and a wick, by means of said wick the substance received in said container is feedable to said electrically actuatable piezo element, wherein, in the assembled state of said container, said wick projects with a first section into the substance received in said container and projects with an opposite free wick end out of said neck of said container;
said housing having a housing bottom wall on its underside in relation to the vertical axis direction, said housing bottom wall having a mounting dome extending upwards in the vertical axis direction and inwards into said housing interior and a mounting opening formed therein on a housing bottom wall side which is accessible from a housing exterior, said mounting opening in said housing interior merges into a wick opening at an end of said mounting dome, which is adjoined, with respect to the vertical axis direction, outwardly and/or upwardly, by said electrically actuable piezo element and also by said housing outlet opening, via which the substance to be discharged escapes from said housing into an environment; and
said mounting dome configured to receive said neck of said container and having a holding device, said neck being introduced into said mounting dome via said mounting opening, at least partially, and said mounting dome holding said neck releasably in said mounting dome by means of said holding device such that said wick extends with said free wick end in the assembled state of said container into and/or through said wick opening and adjoins said electrically actuable piezo element.

38. The piezoelectrically operated dispenser according to claim 37, wherein said mounting dome has a mounting and receiving zone for said electrically actuable piezo element adjoining said wick opening, said mounting and receiving zone is configured to arrange and/or hold said electrically actuable piezo element in an area of said wick opening, so that said wick adjoins said electrically actuable piezo element with said free wick end.

39. The piezoelectrically operated dispenser according to claim 38, wherein:
said mounting and receiving zone has a receiving chamber for said electrically actuable piezo element which, for the substance discharge, is formed with at least one receiving chamber outlet opening, and in said receiving chamber said electrically actuable piezo element is received such that it is displaceable into different positions; and
said mounting and receiving zone has a pretensioning device by means of which said electrically actuable piezo element is pretensionable for contact with the free wick end.

40. The piezoelectrically operated dispenser according to claim 39, wherein said receiving chamber is formed by at least one receiving chamber wall which extends away from said wick opening in the vertical axis direction and at least partially encloses said wick opening, as seen in a circumferential direction of said wick opening.

41. The piezoelectrically operated dispenser according to claim 39, wherein said pretensioning device is formed by an elastic spring element, which acts indirectly or directly on said electrically actuable piezo element and is supported on said mounting dome.

42. The piezoelectrically operated dispenser according to claim 39, wherein:
said receiving chamber has a supporting device; and
said pretensioning device is disposed in said receiving chamber and/or said pretensioning device is supported in a region of said receiving chamber outlet opening by means of said supporting device.

43. The piezoelectrically operated dispenser according to claim 42, wherein:
said supporting device is formed by a fixing sleeve, which is placed on said free end of said mounting dome, and which covers said receiving chamber outlet opening with a sleeve wall; and
said sleeve wall having a sleeve opening formed therein, via which the substance to be dispensed escapes from said receiving chamber, and an opening edge region of said sleeve wall projects into said receiving chamber outlet opening for supporting said pretensioning device.

44. The piezoelectrically operated dispenser according to claim 43, wherein:
said receiving chamber has a receiving chamber inner wall; and
said opening edge region of said sleeve wall projecting into said receiving chamber outlet opening, as seen in the circumferential direction, has at least in some regions a bend directed inwards into said receiving chamber in the direction of the vertical axis, so that between said bend and an associated wall region of said receiving chamber inner wall there is formed a supporting device which is U-shaped in cross section and in which said pretensioning device is received and supported.

45. The piezoelectrically operated dispenser according to claim 44, wherein said housing outlet opening, viewed in the vertical axis direction, is disposed above and/or in alignment with said free wick end and in alignment with said wick opening and said receiving chamber outlet opening and said sleeve opening.

46. The piezoelectrically operated dispenser according to claim 37, wherein said free end of said mounting dome in said housing interior is directly adjacent to said housing outlet opening and/or in that said mounting dome, viewed in the vertical axis direction, extends from said housing bottom wall generally over an entire height of said housing interior to said housing outlet opening.

47. The piezoelectrically operated dispenser according to claim 37, wherein:
said mounting dome has, as seen in the vertical axis direction, an upper dome portion and, in relation thereto, a lower dome portion;
said lower dome portion has said mounting opening and said holding device for said neck of said container and/or extends approximately to a center of said housing interior, as viewed in the vertical axis direction; and
said upper dome portion has said mounting and receiving zone for said electrically actuable piezo element and said wick opening, said wick opening being in a form of a wick channel which has a lower opening area, relative to the vertical axis direction, via which said wick is insertable into said wick channel, and, in contrast thereto, an upper opening area.

48. The piezoelectrically operated dispenser according to claim 47, wherein said free wick end projects beyond said upper opening area in a mounted state and projects into said receiving chamber of said mounting and receiving zone.

49. The piezoelectrically operated dispenser according to claim 47, wherein said wick channel, viewed in the vertical axis direction, directly adjoins said mounting opening of said lower dome portion in an upward direction and/or in that an end face of said neck of said container in the assembled state abuts and/or is supported against a wall portion of said upper dome portion forming said lower opening area of said wick channel.

50. The piezoelectrically operated dispenser according to claim 37, wherein said housing bottom wall forming an underside of said housing surrounds said mounting dome with a bottom wall edge region which is aligned generally horizontally with respect to the vertical axis direction and seen in a circumferential direction.

51. The piezoelectrically operated dispenser according to claim 37, wherein:
said container has a cylindrical circumferential wall with a container bottom wall extending substantially horizontally with respect to the vertical axis direction and a container top wall extending substantially parallel thereto, said container top wall having said neck; and
said housing together with said container, in the assembled state, form a tower-like structure, said housing resting with said housing bottom wall on said container top wall.

52. A method for operating a piezoelectrically operated dispenser, which comprises the steps of:
providing the piezoelectrically operated dispenser according to claim 37, the piezoelectrically operated dispenser further having an activation and deactivation device for activating and deactivating the piezoelectrically operated dispenser and simultaneously also forms a setting device for setting a dispensing rate;
actuating the piezoelectrically operated atomizer system such that:
starting from a switched-off state of the piezoelectrically operated dispenser, a first actuation of the activation and deactivation device for a defined minimum switch-on actuation period switches the piezoelectrically operated dispenser on and sets it to a first operating mode with which a first dispensing rate is set;
subsequently, for each further actuation of the activation and deactivation device which is shorter than a defined minimum switch-off actuation period, setting at least one further operating mode with a higher dispensing rate than a respective previous dispensing rate, and that the activation and deactivation device is switched off in an event of an actuation which exceeds a defined switch-off actuation period;
or
subsequently, for each further actuation of the activation and deactivation device, a defined number of further operating modes, with a higher dispensing rate than the respective previous dispensing rate is set, and that after the actuation of a last operating mode a further actuation of the activation and deactivation device switches off the activation and deactivation device.

53. The method according to claim 52, wherein:
that, in the first operating mode, the spraying time of the dispenser is in each case 1 to 10 seconds, and an interval between individual spraying times is 5 to 30 minutes; and
that in the at least one further operating mode, the spraying time of the dispenser is increased compared to the spraying time of the first operating mode and/or the interval between the individual spraying times is shortened compared to the interval of the first operating mode.

54. The method according to claim 53, wherein:
the piezoelectric atomizer system, after its activation, is operated in an activation mode for a defined activation time, before it automatically switches to a sleeping mode for a remaining time, based on a period greater than the activation time; and
the piezoelectric atomizer system automatically switches back to the activation mode after a time for the sleeping mode has elapsed.

55. The method for operating a dispenser according to claim 52, wherein:
the defined minimum switch-on actuation period lasts from about 0.1 to 3 seconds; and
the defined number of further operating modes is two further operating modes.

56. The method according to claim 53, wherein:
the spraying time of the dispenser is in each case about 3 to 4 seconds; and
the interval between the individual spraying times being 15 to 17 minutes.
